(19) United States
(12) Patent Application Publication
(10) Pub. No.: US 2024/0425966 A1
(43) Pub. Date: Dec. 26, 2024

(54) GAS-ASSISTED COCRYSTAL DE-SUBLIMATION

(71) Applicant: THE REGENTS OF THE UNIVERSITY OF MICHIGAN, Ann Arbor, MI (US)

(72) Inventors: Max Shtein, Ann Arbor, MI (US); Shea Sanvordenker, Ann Arbor, MI (US)

(21) Appl. No.: 18/690,370

(22) PCT Filed: Sep. 9, 2022
(86) PCT No.: PCT/US22/43040

§ 371 Date: Mar. 8, 2024

Related U.S. Application Data
(60) Provisional application No. 63/242,533, filed on Sep. 10, 2021.

(51) Int. Cl. C23C 14/06; A61K 9/70; A61K 31/194; A61K 31/405; A61K 31/55; A61K 47/22; C23C 14/22; C23C 14/58

(52) U.S. Cl. CPC C23C 14/06; A61K 9/7007; A61K 31/194; A61K 31/405; A61K 31/55; A61K 47/22; C23C 14/228; C23C 14/5806

(57) ABSTRACT

Disclosed herein are solvent-free vapor deposition methods comprising: vaporizing a bulk cocrystalline material to form a vapor, entraining the vapor into a carrier gas, and depositing a film comprising the cocrystalline material on one or more discrete regions of a substrate. Also provided are solid films and articles comprising a surface of a solid substrate having one or more discrete regions patterned with a deposited solid film of a cocrystalline material, produced by the solvent-free vapor deposition methods disclosed herein.

### CROSS-REFERENCE TO RELATED APPLICATIONS

Priority is claimed to U.S. Ser. No. 63/242,533, filed Sep. 10, 2021, the disclosures of which are hereby incorporated by reference in their entirety.

### STATEMENT OF GOVERNMENT INTEREST

This invention was made with government support under 2029139 awarded by the National Science Foundation (NSF). The government has certain rights in the invention.

### BACKGROUND

Organic cocrystals are important in pharmaceutical formulation, energetic materials, foods, and other applications (1), (2), (3). The molecular packing and crystal structure of the cocrystal differ from those of the pure ingredients, often yielding a solubility advantage (SA) when compared to the pure ingredient, i.e. achieving a higher dynamic concentration in a biological system than with a pure form (4). Different coformers can be employed to make cocrystals of the same active ingredient with different SAs, melting point, and/or mechanical characteristics to meet the potential needs of an application, without modifying the active ingredient's molecular structure (and therefore, biological action mechanism) (5). Guiding principles and methods have been developed for the selection of coformers and creation of cocrystals (6), (7), (8), (11). These approaches have been limited to generating bulk powders, limiting possible drug delivery methods.

Several sublimation-based formation methods of cocrystal formation have been investigated in recent years (15-18). This includes preparation by transporting individually sublimated API and coformer to a cooled tube with nitrogen carrier gas (17). Cocrystals have also been screened by sublimation of pre-formed cocrystals, along with both API and coformer phase (18). These prior methods rely on sublimation of each cocrystal component separately, followed by co-deposition on a substrate to form a cocrystalline material, or sublimation of cocrystalline material without use of a carrier gas to direct the subsequent deposition of the material.

There remains a need for improved processes in which pharmaceutical cocrystals are sublimed, transported by a carrier gas, and directed at high velocity at a substrate, where nano- and micro-scopic cocrystals are formed, in order to afford “touch-free”, non-mechanical, single-step means of particle size reduction and surface coating for cocrystalline materials.

### SUMMARY

Provided herein are solvent-free vapor deposition methods comprising: vaporizing a bulk cocrystalline material to form a vapor, entraining the vapor into a carrier gas, and depositing a film comprising the cocrystalline material on one or more discrete regions of a substrate.

Also provided are solid films comprising a deposited cocrystalline material, produced by the solvent-free vapor deposition methods disclosed herein.

Further provided are articles comprising: a surface of a solid substrate having one or more discrete regions patterned with a deposited solid film of a cocrystalline material, produced by the solvent-free vapor deposition methods disclosed herein.

### BRIEF DESCRIPTION OF THE DRAWINGS

FIG. 1 shows a diagram of an apparatus suitable for use with the methods disclosed herein.

FIG. 2 shows TGA and DSC results of active pharmaceutical ingredients (APIs), coformers and traditionally formed cocrystal powders for (a) CBZ-SUC and (b) IND-SAC.

FIG. 3 shows SEM of (a) CBZ-SUC cocrystal formed via solvent evaporation, (b) post-ground in agate mortar and (c) de-sublimated CBZ-SUC on glass. 100 μm scale bar.

FIG. 4 shows powder X-ray diffraction (PXRD) of powder and deposit for (a) CBZ-SUC system and (b) IND-SAC system. Arrows indicate characteristic cocrystal peaks, stars and dagger correspond to SUC and CBZ peaks, respectively.

FIG. 5 shows optical micrographs of IND-SAC de-sublimate before and after annealing. De-sublimate sample 1 (a) was annealed at 60° C. for 1 hour (b) and sample 2 (d) was annealed at 120° C. for 1 hour (e). (c) and (f) were taken with a green light filter. 200 μm scale bar.

FIG. 6 shows PXRD of solvent evaporation-formed cocrystals before and after heating to 5° C. above the melt and cooling to 25° C. or 60° C. Isotherms were held for 10 minutes and the heating/cooling rate was 10° C. per minute.

### DETAILED DESCRIPTION

The instant disclosure demonstrates a novel process by which a cocrystal is sublimed into a carrier gas, and impinged at high velocity onto a cooled substrate, where both components of the cocrystal de-sublimate as nano- and microscopic cocrystals. This gas-assisted process enables the formation of cocrystalline coatings that would be challenging to obtain using conventional powder processing methods.

Provided herein are solvent-free vapor deposition methods comprising: vaporizing a bulk cocrystalline material to form a vapor, entraining the vapor into a carrier gas, and depositing a film comprising the cocrystalline material on one or more discrete regions of a substrate. In some cases, vaporizing the cocrystalline material comprises subliming the cocrystalline material to form the vapor. In some cases, vaporizing the cocrystalline material occurs at atmospheric pressure. In some cases, vaporizing the cocrystalline material occurs under reduced pressure. In some cases, entraining of the bulk crystalline material into the carrier gas is conducted by heating a source of a bulk cocrystalline material to sublimate or evaporate the cocrystalline material. In some cases, the carrier gas is substantially free of any solvents prior to the depositing. In some cases, the carrier gas is substantially free of water vapor prior to the depositing. In some cases, prior to the entraining, the bulk cocrystalline material is in a form selected from the group consisting of: a powder, a pressed pellet, and a porous material. In some cases, prior to the entraining, the bulk cocrystalline material is a powder. In some cases, prior to the entraining, the bulk cocrystalline material is a pressed pellet. In some cases, prior to the entraining, the bulk cocrystalline material is a porous material. In some cases, vaporizing occurs with substantially no thermal degradation of the cocrystalline material. In some cases, the bulk cocrystalline material is volatilized in a stoichiometric ratio. In some cases, the deposited film comprises crystalline or polycrystalline cocrystalline material. In some cases, the deposited film comprises crystalline cocrystalline material. In some cases, the deposited film comprises polycrystalline cocrystalline material. In some cases, the deposited film has an average crystal size greater than or equal to about 2 nm to less than or equal to about 200 nm. In some cases, the film comprises the cocrystalline material as nanoscopic or microscopic crystals. In some cases, the film comprises the cocrystalline material as nanoscopic crystals. In some cases, the film comprises the cocrystalline material as microscopic crystals. In some cases, the crystals have a major dimension of greater than or equal to about 5 nm to less than or equal to about 10 μm. In some cases, the crystals have an average volume of 10 μm³ or smaller. In some cases, the cocrystalline material comprises two or more different molecular compounds in a stoichiometric ratio. In some cases, the stoichiometry of the bulk cocrystalline material is substantially the same as the stoichiometry of the film comprising the cocrystalline material. In some cases, depositing the film comprises impinging the vapor onto a substrate warmed to above 25° C. In some cases, depositing the film comprises impinging the vapor onto a cooled substrate. In some cases, the substrate is cooled to a temperature at or below the freezing point of the cocrystalline material. In some cases, the solvent-free vapor deposition method further comprises an annealing step. In some cases, the annealing step comprises maintaining the film deposited on the substrate at a temperature higher than the printing temperature for a specified time duration. In some cases, the annealing step comprises maintaining the film deposited on the substrate at 60° C. or higher. In some cases, the annealing step comprises maintaining the film deposited on the substrate at 120° C. or higher. In some cases, the annealing step lasts at least 30 minutes. In some cases, the annealing step lasts about 30 minutes. In some cases, the annealing step lasts at least 60 minutes. In some cases, the annealing step lasts about 60 minutes. In some cases, the deposited cocrystalline material comprises a pharmaceutical active ingredient or a new chemical entity selected from the group consisting of: antiproliferative agents; anti-rejection drugs; anti-thrombotic agents; anti-coagulants; antioxidants; free radical scavengers; nucleic acids; saccharides; sugars; nutrients; hormones; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; and combinations thereof. In some cases, the cocrystalline material comprises carbamazepine, succinic acid, indomethacin, saccharine, and combinations thereof. In some cases, the cocrystalline material comprises carbamazepine. In some cases, the cocrystalline material comprises succinic acid. In some cases, the cocrystalline material comprises indomethacin. In some cases, the cocrystalline material comprises saccharine. In some cases, In some cases, the cocrystalline material comprises carbamazepine and succinic acid. In some cases, the cocrystalline material comprises indomethacin and saccharine.

Also provided are solid films comprising a deposited cocrystalline material, produced by the solvent-free vapor deposition methods disclosed herein. In some cases, the deposited cocrystalline compound in the solid film is crystalline or polycrystalline. In some cases, the deposited cocrystalline compound in the solid film is crystalline. In some cases, the deposited cocrystalline compound in the solid film is polycrystalline. In some cases, the deposited film has an average crystal size greater than or equal to about 2 nm to less than or equal to about 200 nm. In some cases, the cocrystalline material comprises a compound selected from the group consisting of: anti-proliferative agents; anti-rejection drugs; anti-thrombotic agents; anticoagulants; antioxidants; free radical scavengers; nucleic acids; saccharides; sugars; nutrients; hormones; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; and combinations thereof. In some cases, the cocrystalline material comprises carbamazepine, succinic acid, indomethacin, saccharine, and combinations thereof. In some cases, the cocrystalline material comprises carbamazepine. In some cases, the cocrystalline material comprises succinic acid. In some cases, the cocrystalline material comprises indomethacin. In some cases, the cocrystalline material comprises saccharine. In some cases, In some cases, the cocrystalline material comprises carbamazepine and succinic acid. In some cases, the cocrystalline material comprises indomethacin and saccharine.

Also provided are articles comprising a surface of a solid substrate having one or more discrete regions patterned with a deposited solid film of a cocrystalline material, produced by the solvent-free vapor deposition method. In some cases, the deposited cocrystalline compound in the solid film is crystalline or polycrystalline. In some cases, the deposited cocrystalline compound in the solid film is crystalline. In some cases, the deposited cocrystalline compound in the solid film is polycrystalline. In some cases, the deposited film has an average crystal size greater than or equal to about 2 nm to less than or equal to about 200 nm. In some cases, the cocrystalline material comprises a compound selected from the group consisting of: anti-proliferative agents; anti-rejection drugs; anti-thrombotic agents; anticoagulants; antioxidants; free radical scavengers; nucleic acids; saccharides; sugars; nutrients; hormones; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; and combinations thereof. In some cases, the cocrystalline material comprises carbamazepine, succinic acid, indomethacin, saccharine, and combinations thereof. In some cases, the cocrystalline material comprises carbamazepine. In some cases, the cocrystalline material comprises succinic acid. In some cases, the cocrystalline material comprises indomethacin. In some cases, the cocrystalline material comprises saccharine. In some cases, In some cases, the cocrystalline material comprises carbamazepine and succinic acid. In some cases, the cocrystalline material comprises indomethacin and saccharine.

### Example 1: Preparation of Cocrystalline Films

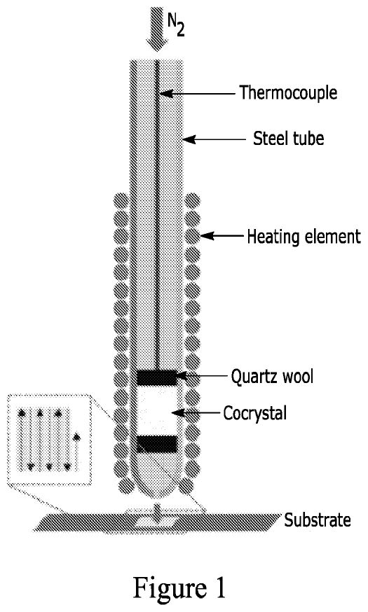

An exemplary apparatus for practicing the solvent-free vapor deposition methods disclosed herein is depicted in the apparatus schematic in FIG. 1. In this example, a steel tube ˜10 mm in diameter with a 1 mm diameter orifice on the downstream end contained pre-formed cocrystal, kept at the desired temperature. Pure nitrogen gas was admitted through the tube at a precisely controlled rate using a Sierra Instruments Smart-Trak 2 Digital Mass Flow Controller, picking up the vapor of the two components of the cocrystal, and being directed at a cooled substrate, while the substrate moved in a raster pattern as shown. The cocrystals loaded into the apparatus as test-cases comprised carbamazepine-succinic acid (CBZ-SUC) and indomethacin-saccharine (IND-SAC), pre-synthesized following previously reported procedures using the solvent evaporation method (19,20).

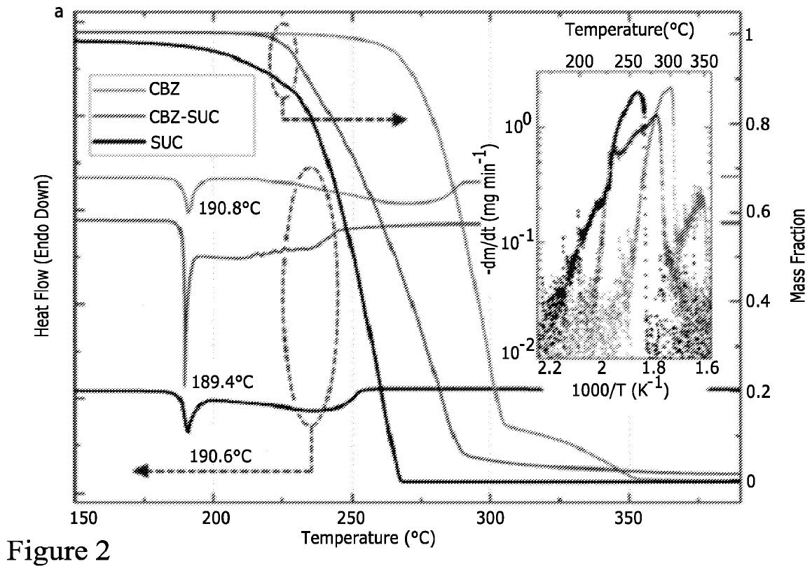

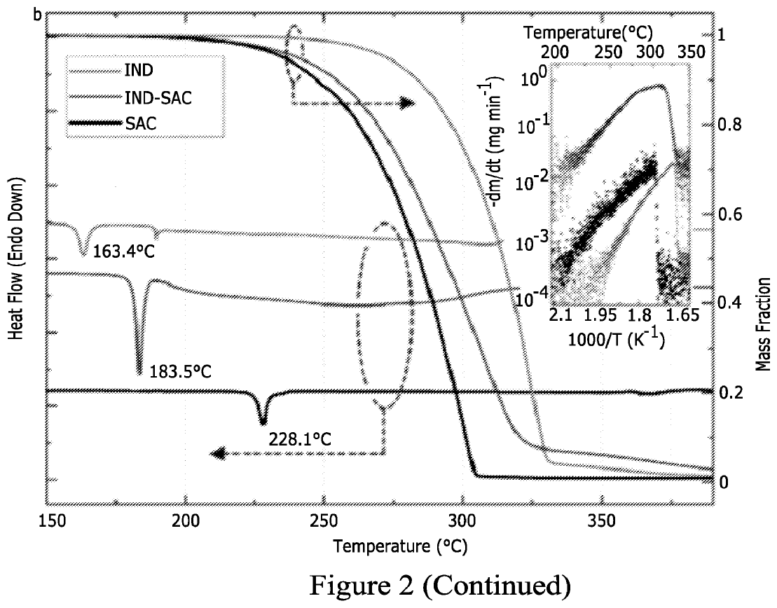

To determine optimal process temperature, differential scanning calorimetry (DSC) and thermogravimetric analysis (TGA) were performed on the pure components, as well as on the cocrystal, shown in FIG. 2. A TA instruments DSC Q200 was used with a 40-400° C. heating range and 10° C. per minute heating rate. Only initial heating curves were collected and analyzed, without a cooling and secondary heating step. The initial melting peak temperature and enthalpy of melting values for each sample was determined using Universal Analysis software from TA instruments. A TA Instruments TGA Q500 was used with a 25-400° C. heating range and a 10° C. per minute heating rate. Powder was placed into a 5 mm diameter aluminum sample pan centered in a platinum hang pan. Enthalpies of sublimation were determined by plotting the logarithm of mass loss rate (−dm/dt) versus inverse temperature (1000/T). Linear sections of the plot indicated classical Arrhenius behavior, where the slope is ΔH/R (ΔH is the enthalpy of vaporization or sublimation, and R is the universal gas constant).

Key parameters from the TGA and DSC data are summarized in Table 1, which agree with published values for the pure and cocrystal species (19-22). The TGA curves for both systems show that the cocrystals exhibit vapor generation in a range of temperatures where material is not thermally degraded, serving as the preferred range of process temperature (Table 2).

TABLE 1
DSC and TGA results.

| | Melting | | | Sublimation | | |
|---|---|---|---|---|---|---|
| | Tₒₙₛₑₜ | Tₚₑₐₖ | ΔHₘₑₗₜ | ΔH_{sub} | T_{low} | T_{high} |
| Material | (° C.) | (° C.) | (kJ/mol) | (kJ/mol) | (° C.) | (° C.) |
| CBZ | 189.2 | 190.8 | 26.5 | 203.3 | 220 | 280 |
| SUC | 187.9 | 190.6 | 33.0 | 96.5 | 140 | 230 |
| CBZ-SUC | 189.1 | 189.4 | 94.6 | 282.7 | 200 | 230 |
| CBZ-SUC | | 187.4 | | | | |
| Print | | | | | | |
| IND | 160.2 | 163.4 | 33.5 | 114.2 | 200 | 320 |
| SAC | 225.4 | 228.1 | 36.3 | 107.0 | 200 | 290 |
| IND-SAC | 181.7 | 183.5 | 73.7 | 102.4 | 200 | 280 |

TABLE 2
Process settings used to generate de-sublimate.

| | | |
|---|---|---|
| Process Parameter | CBZ-SUC | IND-SAC |

| | | | | |
|---|---|---|---|---|
| Mass Loaded | 100 | mg | 200 | mg |
| Cocrystal Temperature | 170° | C. | 200° | C. |
| Nitrogen Flow Rate | 150 | SCCM | 200 | SCCM |
| Separation Distance | 2 | mm | 4 | mm |
| Substrate Temperature | ~25° | C. | 20° | C. |
| Raster Velocity | 0.32 | mm/s | 0.32 | mm/s |

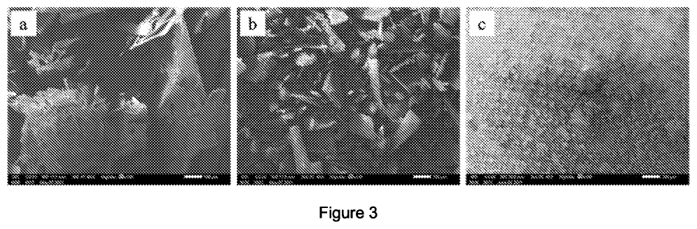

The process was found to largely maintain the stoichiometric ratio of the components and the cocrystalline nature of the material, yet resulted in substantially smaller crystals. For example, scanning electron micrographs (SEMs), collected on a JEOL IT500 SEM and shown in FIG. 3, clearly show the prevalence of large (>100 μm) crystallites in bulk synthesized and dried CBZ-SUC, and slight reduction and uniformization of size after manual grinding in an agate mortar. The gas-processed material, however, exhibits a dramatic reduction in particle size to well below 10 μm without any mechanical impaction of the particles during the process.

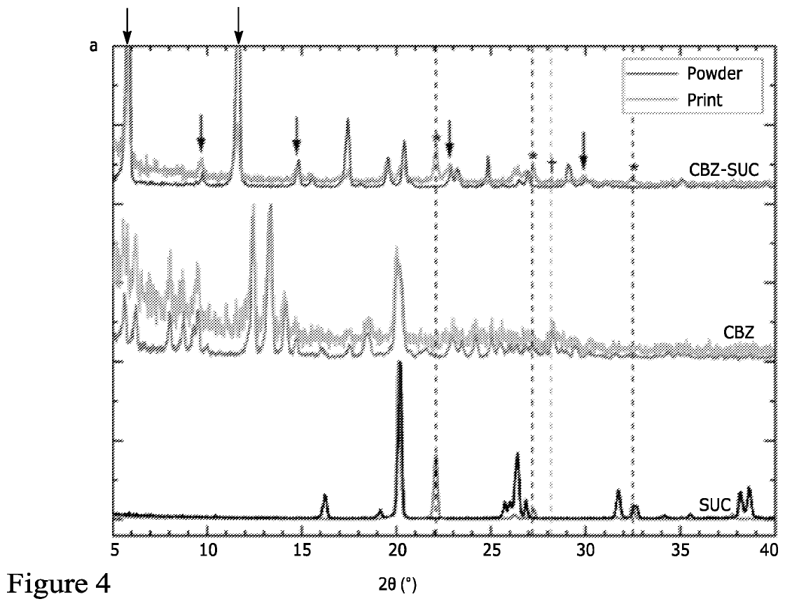

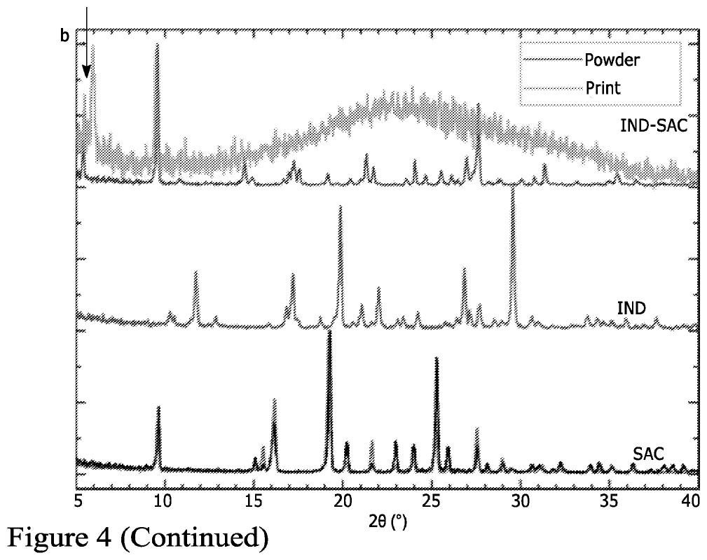

Powder X-Ray diffraction (PXRD) measurements were collected at room temperature (˜300 K), on a Rigaku Miniflex 600 with a CuKα X-ray radiation source (λ=1.54 Å), at a fixed tube voltage of 40 kV and a fixed tube current of 15 mA. Diffraction patterns were recorded for polycrystalline thin films on glass and powders with the beam scanned between 5-40° (20). Peak positions were compared to known peak positions for pure components and cocrystals to identify the presence of any new crystalline phases after de-sublimation. The as-deposited films of CBZ-SUC exhibited crystallinity, retaining the key peaks at 2θ=5.8°, 9.7°, 11.5°, 14.7°, 22.8° and 29.9° previously identified with the cocrystal state (FIG. 4a) (19,23) Four new minor peaks appeared as well, corresponding to the de-sublimated β phase of succinic acid (2θ=22.1°, 27.2°,) 32.5° (24) and Form I of carbamazepine (2θ=28.2°) (25).

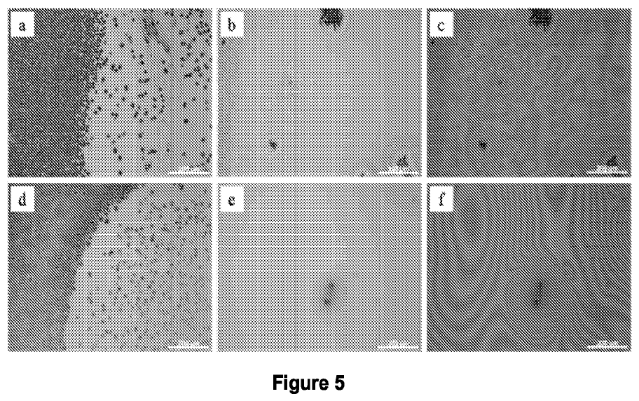

As-deposited films of IND-SAC were amorphous, but began to convert to cocrystals upon annealing (e.g. at 60° and 120° C.), as XRPD data (FIG. 4b) and optical micrographs (FIG. 5) show. An identifying peak of the cocrystal phase at 2θ=5.4° appeared after annealing at 120° C. (20). As-deposited SAC remained in the same phase as the powder.

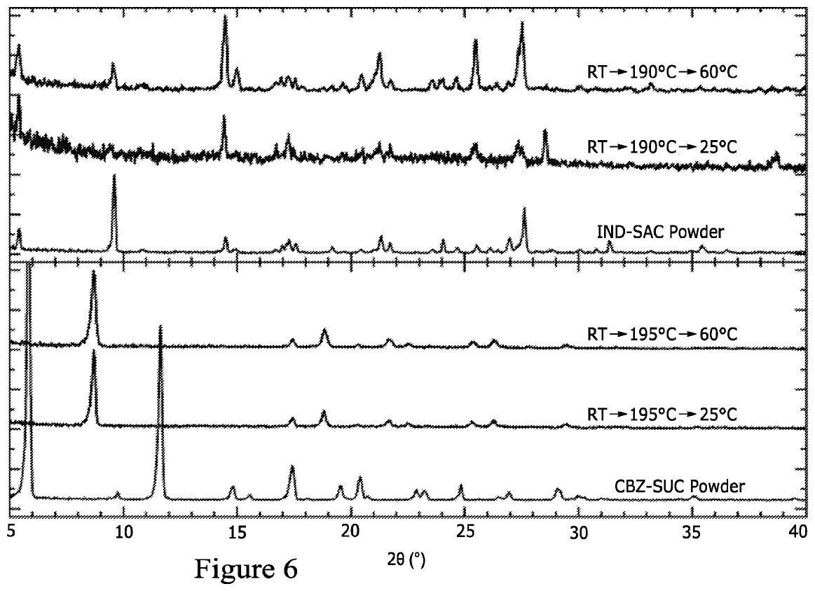

During the process, CBZ-SUC was heated to below its melting point for printing. In contrast, it was necessary to heat IND-SAC above its melting point to allow for sufficient deposition rates during printing. To determine if the cocrystals melt congruently or incongruently, PXRD was performed on melted and cooled samples. PXRD of melted IND-SAC in FIG. 6 indicates it remains a cocrystal once cooled. The characteristic peak at 5.4° is present, as well as all other peaks corresponding to solvent-evaporated formed cocrystal. This suggests the cocrystal does not separate into individual liquid phases upon melting, therefore suggesting that the components volatilize in a stoichiometric ratio.

### Interpretation

Thermal analysis provides a good basis to determine the process window for successful cocrystal de-sublimation. The target temperature for the process is preferably in a region where sublimed API and conformer readily remain in the vapor phase. Too low, and the less volatile compound may de-sublimate within the heated tube, altering the ratio of API to conformer reaching a cooled surface (26). Moreover, decreasing temperature reduces sublimation rate and thus restricts the amount of material that can be processed. The upper temperature limit of the process is largely dictated by the thermal stability of each component. For example, CBZ may begin to decompose appreciably before reaching 180° C., limiting the heating temperature to well below its melting temperature. These factors result in a relatively slim heating window of 160-170° C. for CBZ-SUC. On the other hand, IND is stable up to 248° C., well above its melting temperature (27). To achieve similar de-sublimation rates as CBZ-SUC, heating to above the melting point was necessary. The optimal heating range was determined to be 200-225° C. Temperatures above 225° C. may be viable as well, as indicated by the linear nature of the Arrhenius plot (i.e., logarithmic mass loss rate versus inverse temperature) in that temperature region (FIG. 2b).

Substrate temperature also plays an important role in successful cocrystal de-sublimation. For CBZ-SUC, substrate temperatures between 10° C. and 25° C. were tried, however better success was found with higher-than-room temperatures; IND-SAC was found to de-sublimate into an amorphous solid, requiring an annealing step to crystallize into IND-SAC. A broader range of temperature control may be advantageous for achieving single-step de-sublimation of cocrystals with similar behavior to IND-SAC.

### CONCLUSIONS

The preceding methods and examples provide means for creating organic cocrystal coatings consisting of micronized crystals through a “touch-free”, gas flow-assisted, vapor-based approach, without the need of any mechanical grinding or solvents in the formation of the coating. This technique opens the door to single-step processing and deposition of cocrystal systems to broaden the choice of feasible drug delivery.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

### REFERENCES

1 Shan N et al., Drug Discov. Today 2008, 13 440.
2 Schultheiss N et al., Cryst Eng Comm 2010, 12, 2436.
3 Bolton O et al., Angew Chemie Int Ed. 2011, 50, 8960
4 Good D J et al., Cryst. Growth Des. 2009, 9, 2252.
5 Duggirala N K et al., Chem Commun. 2016, 52, 640.
6 Wood P A et al., Cryst Eng Comm. 2014, 16, 5839.
7 Friščić T et al., Cryst. Growth Des. 2009, 9, 1621.
8 Karimi-Jafari M et al., Cryst. Growth Des. 2018, 18, 6370.
9 Perlovich G L., Cryst Eng Comm., 2015, 17, 7019.
10 Childs S L et al., Mol Pharm., 2007, 4, 323.
11 Rodríguez-Hornedo N et al., Mol. Pharm., 2006, 3, 362.
12 Shalev O et al., Nature Commun., 2017, 8, 711.
13 Shalev O et al., Org. Electron., 2013, 14, 94.
14 Trask A V et al., Cryst. Growth Des. 2005, 5, 1013.
15 M. B. Alsirawan et al., Crystal Growth & Design, vol. 20, no. 9, pp. 6024-6029, 2020.
16 C. O'Malley, A. et al., Chemical Communications, vol. 56, no. 42, pp. 5657-5660 April 2020.
17 T. Zhang, Q. et al., Journal of Crystal Growth, vol. 469, pp. 114-118, 2017.
18 J. Lombard et al., Crystal Growth & Design, vol. 20, no. 12, pp. 7840-7849, 2020.
19 S. L. Childs et al., Cryst Eng Comm, vol. 10, no. 7, p. 856, 2008.
20 S. Basavoju et al., Pharmaceutical Research, vol. 25, no. 3, pp. 530-541, 2008.
21 F. A. Rahman et al., International Journal of Chemical Engineering and Applications, vol. 8, no. 2, pp. 136-140, 2017.
22 A. Fuliaş et al., Journal of Thermal Analysis and calorimetry, vol. 121, no. 3, pp. 1081-1086, 2015.
23 M. Ullah et al., Drug development and industrial pharmacy. vol. 42, no. 6, pp. 969-976, 2016.
24 Q. Yu et al., Journal of Crystal Growth, vol. 340, no. 1, pp. 209-215, 2012.
25 C. Rustichelli et al., Journal of Pharmaceutical and Biomedical Analysis, vol. 23, no. 1, pp. 41-54, 2000.
26 A. N. Manin et al., The Journal of Physical Chemistry B, vol. 118, no. 24, pp. 6803-6814, 2014.
27 M. Rusu et al., Journal of Pharmaceutical and Biomedical Analysis, vol. 24, no. 1, pp. 19-24, 2000.

What is claimed is:

1. A solvent-free vapor deposition method comprising:
vaporizing a bulk cocrystalline material to form a vapor, entraining the vapor into a carrier gas, and depositing a film comprising the cocrystalline material on one or more discrete regions of a substrate.

2. The solvent-free vapor deposition method of claim 1, wherein vaporizing the cocrystalline material comprises subliming the cocrystalline material to form the vapor.

3. The solvent-free vapor deposition method of claim 1 or 2, wherein vaporizing the cocrystalline material occurs at atmospheric pressure.

4. The solvent-free vapor deposition method of claim 1 or 2, wherein vaporizing the cocrystalline material occurs under reduced pressure.

5. The solvent-free vapor deposition method of any one of claims 1 to 4, wherein the entraining of the bulk crystalline material into the carrier gas is conducted by heating a source of a bulk cocrystalline material to sublimate or evaporate the cocrystalline material.

6. The solvent-free vapor deposition method of any one of claims 1 to 5, wherein the carrier gas is substantially free of any solvents prior to the depositing.

7. The solvent-free vapor deposition method of any one of claims 1 to 6, wherein prior to the entraining, the bulk cocrystalline material is in a form selected from the group consisting of: a powder, a pressed pellet, and a porous material.

8. The solvent-free vapor deposition method of any one of claims 1 to 7, wherein vaporizing occurs with substantially no thermal degradation of the cocrystalline material.

9. The solvent-free vapor deposition method of any one of claims 1 to 8, wherein the bulk cocrystalline material is volatilized in a stoichiometric ratio.

10. The solvent-free vapor deposition method of any one of claims 1 to 9, wherein the deposited film comprises crystalline or polycrystalline cocrystalline material.

11. The solvent-free vapor deposition method of 10, wherein the deposited film has an average crystal size greater than or equal to about 2 nm to less than or equal to about 200 nm.

12. The solvent-free vapor deposition method of claim 10, wherein the film comprises the cocrystalline material as nanoscopic or microscopic crystals.

13. The solvent-free vapor deposition method of claim 12, wherein the crystals have a major dimension of greater than or equal to about 5 nm to less than or equal to about 10 μm.

14. The solvent-free vapor deposition method of claim 13, wherein the crystals have an average volume of 10 μm³ or smaller.

15. The solvent-free vapor deposition method of any one of claims 1 to 14, wherein the cocrystalline material comprises two or more different molecular compounds in a stoichiometric ratio.

16. The solvent-free vapor deposition method of any one of claims 1 to 15, wherein the stoichiometry of the bulk cocrystalline material is substantially the same as the stoichiometry of the film comprising the cocrystalline material.

17. The solvent-free vapor deposition method of any one of claims 1 to 16, wherein depositing the film comprises impinging the vapor onto a substrate warmed to above 25° C.

18. The solvent-free vapor deposition method of any one of claims 1 to 16, wherein depositing the film comprises impinging the vapor onto a cooled substrate.

19. The solvent-free vapor deposition method of claim 18, wherein the substrate is cooled to a temperature at or below the freezing point of the cocrystalline material.

20. The solvent-free vapor deposition method of any one of claims 1 to 19, further comprising an annealing step.

21. The solvent-free vapor deposition method of claim 20, wherein the annealing step comprises maintaining the film deposited on the substrate at a temperature higher than the printing temperature for a specified time duration.

22. The solvent-free vapor deposition method of claim 21, wherein the annealing step comprises maintaining the film deposited on the substrate at 60° C. or higher.

23. The solvent-free vapor deposition method of claim 22, wherein the annealing step comprises maintaining the film deposited on the substrate at 120° C. or higher.

24. The solvent-free vapor deposition method of any one of claims 20 to 23, wherein the annealing step lasts at least 30 minutes.

25. The solvent-free vapor deposition method of claim 24, wherein the annealing step lasts at least 60 minutes.

26. The solvent-free vapor deposition method of any one of claims 1 to 25, wherein the deposited cocrystalline material comprises a pharmaceutical active ingredient or a new chemical entity selected from the group consisting of: antiproliferative agents; anti-rejection drugs; anti-thrombotic agents; anti-coagulants; antioxidants; free radical scavengers; nucleic acids; saccharides; sugars; nutrients; hormones; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; and combinations thereof.

27. The solvent-free vapor deposition method of any one of claims 1 to 26, wherein the cocrystalline material comprises carbamazepine, succinic acid, indomethacin, saccharine, and combinations thereof.

28. A solid film comprising a deposited cocrystalline material, produced by the solvent-free vapor deposition method of any one of claims 1 to 27.

29. The solid film of claim 29, wherein the deposited cocrystalline compound in the solid film is crystalline or polycrystalline.

30. The solid film of claim 29, having an average crystal size greater than or equal to about 2 nm to less than or equal to about 200 nm.

31. The solid film of any one of claims 28 to 30, wherein the cocrystalline material comprises a compound selected from the group consisting of: anti-proliferative agents; anti-rejection drugs; anti-thrombotic agents; anticoagulants; antioxidants; free radical scavengers; nucleic acids; saccharides; sugars; nutrients; hormones; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; and combinations thereof.

32. The solid film of any one of claims 28 to 31, wherein the deposited cocrystalline material comprises carbamazepine, succinic acid, indomethacin, saccharine, and combinations thereof.

33. An article comprising:
a surface of a solid substrate having one or more discrete regions patterned with a deposited solid film of a cocrystalline material, produced by the solvent-free vapor deposition method of any one of claims 1 to 27.

34. The article of claim 33, wherein the deposited cocrystalline material is crystalline or polycrystalline.

35. The article of claim 34, having an average crystal size greater than or equal to about 2 nm to less than or equal to about 200 nm.

36. The article of any one of claims 33 to 35, wherein the deposited cocrystalline material comprises a pharmaceutical active ingredient or a new chemical entity selected from the group consisting of: anti-proliferative agents; anti-rejection drugs; anti-thrombotic agents; anti-coagulants; antioxidants; free radical scavengers; nucleic acids; saccharides; sugars; nutrients; hormones; cytotoxin; hormonal agonists; hormonal antagonists; inhibitors of hormone biosynthesis and processing; antigestagens; antiandrogens; anti-inflammatory agents; nonsteroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; and combinations thereof.

37. The article of claim 36, wherein the deposited cocrystalline material comprises carbamazepine, succinic acid, indomethacin, saccharine, and combinations thereof.
